(19) United States
(12) Patent Application Publication
(10) Pub. No.: US 2020/0393333 A1
(43) Pub. Date: Dec. 17, 2020

(54) WATER QUALITY MONITORING DEVICE AND SYSTEM THEREOF

(71) Applicant: King Young Technology Co., Ltd., Taipei City (TW)

(72) Inventor: Ta-Yang Cheng, Taipei City (TW)

(73) Assignee: King Young Technology Co., Ltd., Taipei City (TW)

(21) Appl. No.: 16/438,490

(22) Filed: Jun. 12, 2019

(51) Int. Cl. G01N 1/10; G01N 33/18

(52) U.S. Cl. CPC G01N 1/10; G01N 33/18

(57) ABSTRACT

The present invention discloses a water quality monitoring device and system. By revealing at least a part of a tube above the water surface, water quality data detected below the water surface is transmitted. The water quality monitoring device sequentially includes, from top to bottom, an upper tube, a first connecting member and a middle tube. The water quality monitoring device appearing slightly long tubular further consists of a signal transceiving module in the upper tube, a monitor in the middle tube, and a sensing portion protruding out of the middle tube. A water quality monitoring system can further be formed by connecting a plurality of water quality monitoring devices to a network to monitor water quality conditions at a remote end, thereby monitoring water quality conditions in real time.

### BACKGROUND OF THE INVENTION

### Field of the Invention

The present invention relates to a water quality monitoring device and system, and more particularly to a water quality monitoring device and system capable of monitoring water quality conditions below a water surface of an aquaculture farm.

### Description of the Prior Art

In a traditional approach for monitoring water quality conditions of an aquaculture farm, aquaculture industrialists usually need to be present at an aquaculture farm and sample water for subsequent inspection or inspect water samples by using handheld apparatuses.

For aquaculture farms in a large number of in an extensive area, a substantial time is required for water sampling, which is both time and effort consuming and is thus an extremely inconvenient processing approach with respect to aquaculture industrialists.

Furthermore, because sampling can only be performed by being present at an aquaculture farm in order to learn current water quality conditions, appropriate processing time points may be delayed, leading to pathological changes or even deaths of aquatic living organisms and hence unnecessary losses.

### SUMMARY OF THE INVENTION

It is an object of the present invention to enable aquaculture industrialists to conveniently obtain water quality monitoring data.

It is another object of the present invention to reduce possible losses for aquaculture industrialists.

To achieve the above and other objects, the present invention provides a water quality monitoring device including an upper tube, a first connecting member, a middle tube, a monitor and a signal transceiving module. The first connecting member has an upper end thereof connected to an opening at a lower end of the upper tube, and has a lower end thereof connected to an opening at an upper end of the middle tube. The monitor includes a signal transmission wire sequentially passing through the interior of the middle tube and the first connecting member. The monitor is in either one of two configurations of: configured at a lower end of the interior of the middle tube and extending out of the opening at the lower end of the middle tube, or configured at an exterior of the middle tube and connecting to one end of the signal transmission wire, wherein the end of the signal transmission wire is fixed at the lower end of the interior of the middle tube. The signal transceiving module is installed at an interior of the upper tube and coupled to the signal transmission wire, and is for monitoring water quality conditions at a remote end by using transmission of wireless signals.

In one embodiment of the present invention, a sealing ring is further included between the first connecting member and the middle tube, and an accommodating space is defined at the interior of the middle tube by the first connecting member and the monitor configured at the lower end of the middle tube, thus preserving gas in the accommodating space therein.

In one embodiment of the present invention, a lower tube and a second connecting member are further included. The lower tube has an opening at an upper end thereof, at least one window connecting to an interior thereof on a tube wall thereof, and a dilated opening matching an outer diameter of the middle tube at the upper end thereof. The second connecting member has an upper end thereof connected to a lower end of the lower tube.

In one embodiment of the present invention, further comprising: a counterweight block, being in at least one of six configurations of: in the middle tube, outside the middle tube, in the lower tube, outside the lower tube, in the second connecting member and outside the second connecting member.

In one embodiment of the present invention, a position at which the counterweight block is configured causes a center of gravity of the water quality monitoring system to locate below a middle of the water quality monitoring device, and simultaneously causes an antenna portion of the signal transceiving module to be revealed above a water surface when the water quality monitoring device is deployed in water.

In one embodiment of the present invention, the lower tube comprises a corresponding net mask covering the at least one window.

In one embodiment of the present invention, the monitor comprises at least one sensing unit, and the at least one sensing unit is at least one selected from a temperature sensing unit, an electrical conductivity sensing unit, an oxidation-reduction potential sensing unit, a dissolved oxygen sensing unit, a pH value sensing unit and an ion-selective electrode sensing unit.

In one embodiment of the present invention, further comprising a solar power module provided at an upper side or around a tube of the upper tube, and the solar power module is coupled to the signal transmission wire.

In one embodiment of the present invention, further comprising an end tube having a pointed portion at a lower end thereof, an upper end of the end tube connected to a lower end of the second connecting member.

In one embodiment of the present invention, the second connecting member is connected with an anchor member.

To achieve the above and other objects, the present invention further provides a water quality monitoring system, which is connected to a remote server to provide water quality data to a terminal device. The water quality monitoring system includes the foregoing water quality monitoring device and a wireless router. The wireless router wirelessly is connected to the water quality monitoring devices to obtain the water quality data monitored by the monitors of the water quality monitoring devices.

In one embodiment of the present invention, the signal transceiving modules of the water quality monitoring devices are radio-frequency (RF) transceiving modules to thereby establish RF communication with the wireless router.

To achieve the above and other objects, the present invention further provides a water quality monitoring system, which is connected to a remote server to provide water quality data to a terminal device. The water quality monitoring system includes foregoing water quality monitoring device, wherein the signal transceiving modules of the water quality monitoring devices are signal transceiving modules compliant to a mobile communication technology standard (e.g. the fourth generation or fifth generation of mobile communication technology standard, or a communication technology standard of a later version).

Accordingly, using a water quality monitoring device appearing slightly long tubular floating in the water of an aquaculture farm, current water quality data can be transmitted in real time, enabling an aquaculture industrialist to conveniently obtain water quality monitoring data and to take corresponding measures in real time before or while a situation occurs, thus reducing losses and achieving more effective protection.

### BRIEF DESCRIPTION OF THE DRAWINGS

FIG. 1 is an exploded schematic diagram of a water quality monitoring device according to an embodiment of the present invention;

FIG. 2 is an exploded schematic diagram of a water quality monitoring device according to another embodiment of the present invention;

FIG. 3 is an assembly diagram of a water quality monitoring device according to an embodiment of the present invention; and

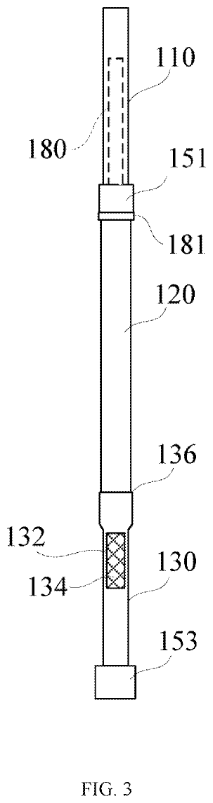

FIG. 4 is a schematic diagram of a water quality monitoring system according to an embodiment of the present invention.

### DETAILED DESCRIPTION OF THE INVENTION

Objectives, features, and advantages of the present disclosure are hereunder illustrated with specific embodiments, depicted with drawings, and described below.

Words, such as “comprise”, “include”, “have” and any equivalent thereof, used herein are not restricted to elements disclosed herein. Instead, the words may be descriptive of any elements which are not expressly disclosed herein but are required for the components, structures, devices, modules, and systems.

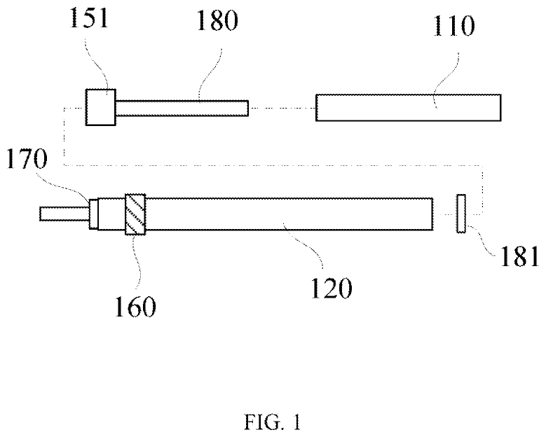

FIG. 1 shows an exploded schematic diagram of a water quality monitoring device according to an embodiment of the present invention. The water quality monitoring device 100 sequentially includes, from top to bottom, an upper tube 110, a first connecting member 151 and a middle tube 120. The water quality monitoring device 100 may further be provided with a monitor 170 and a signal transceiving module 180. As shown in FIG. 1, the upper tube 110 and the middle tube 120 are connected by the first connecting member 151. The upper tube 110 has an opening at a lower end thereof, and the middle tube 120 has an opening at each of upper and lower ends thereof. The assembled water quality monitoring device 100 appears as a slightly long tubular tube.

The monitor 170 can be configured at the lower end of an interior of the middle tube 120 and protruded outside the opening at the lower end of the middle tube 120. Thus, the opening at the lower end of the middle 120 can be at the same time sealed by the monitor 170. The part of the monitor 170 protruding out of the opening at the lower end of the middle tube 120 is a main sensing portion, which is in contact with a water flow to detect water quality conditions.

The signal transceiving module 180 is installed at an interior of the upper tube 110. The signal transceiving module 180 is coupled to the monitor 170 by a signal transmission wire (not shown) passing through the interior of the middle tube 120 and the first connecting member 151.

On the other hand, although not shown in FIG. 1, the monitor 170 can also be configured at an exterior of the middle tube 120, and one end of the signal transmission wire can be fixed at the lower end of the interior of the middle tube 120. Thus, the opening at the lower end of the middle tube 120 can be sealed by using a fixing portion at one end of the signal transmission wire. Moreover, with one end of the signal transmission wire extending out of the opening at the lower end of the middle tube 120, the monitor 170 can be suspended by that end of the signal transmission wire. For example, the monitor 170 is a probe connected to an end of the signal transmission wire, and a distance by which the monitor 170 is extended can be determined by extending the signal transmission wire.

As shown in FIG. 1, a counterweight block 160 can be configured at an outer periphery near the lower end of the middle pipe 120. The counterweight block 160 allows an upper part of the water quality monitoring device 100 to stay afloat on the water surface without tilting. Further, in other embodiments, the counterweight block 160 ca also be configured in the middle tube 120, or the counterweight block 160 can simultaneously be configured at both the interior and exterior of the middle tube 120. A sealing ring 182 (e.g., an O-ring) can be further included between the first connecting member 150 and the middle tube 120. Meanwhile, with the first connecting member 151 and the monitor 170 configured at the lower end of the middle tube 120, an accommodating space can be defined at the interior of the middle tube 120, so as to preserve gas or a filler in the accommodating space therein. The sealing ring 182 further enhances airtightness between the first connecting member 151 and the middle tube 120.

The counterweight block 160 also allows a transceiving antenna in the signal transceiving module 180 to be revealed from the water surface, so as to prevent radio waves emitted therefrom being absorbed by water. A position at which the counterweight block 160 is configured causes the center of gravity of the water quality monitoring device 100 to locate below the middle of the water quality monitoring device 100, and at the same time allows an antenna portion of the signal transceiving module 180 to be revealed above the water surface when the water quality monitoring device 100 is deployed in water.

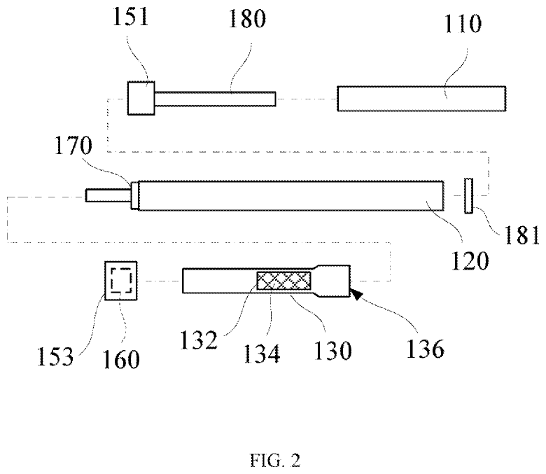

FIG. 2 shows an exploded schematic diagram of a water quality monitoring device according to another embodiment of the present invention. The water quality monitoring device 100 sequentially includes, from top to bottom, an upper tube 110, a first connecting member 151, a middle tube 120, a lower tube 130 and a second connecting member 153. The water quality monitoring device 100 can be further provided with a counterweight block 160, a monitor 170 and a signal transceiving module 180.

As shown in FIG. 2, the lower tube 130 has an opening at an upper end thereof, and at least one window 132 connecting to an interior thereof on a tube wall thereof. The lower tube 130 has, at an upper end thereof, a dilated opening 136 matching with an outer diameter of the middle tube 120. Airtightness is achieved by the dilated opening 136 and the opening at the lower end of the middle tube 120. The second connecting member 153 has an upper end thereof connected to a lower end of the lower tube 130.

As shown in FIG. 2, the monitor 170 can be fixed at the lower end of the middle tube 120, such that the monitor 170 is installed in the middle tube 120, while the projecting sensing portion protrudes outside the middle tube 120. The projecting sensing portion of the monitor 170 can correspond to the window 132 (only one window is shown in FIG. 2) on the lower tube 130. The window 132 on the lower tube 130 is designed in a way that the water flow enters the lower tube 130 and water quality conditions thereof can be detected by the sensing portion of the monitor 170. Further, a net mask 134 covering the window 132 can be further included on the window 132 to prevent any alien object from entering the lower tube 130 and causing congestion, and also to prevent invasion or pollution of living organisms in the water from causing damage of the sensing portion.

The monitor 170 measures water quality conditions in the water, and can be simultaneously provided with a plurality of sensing units to provide various kinds of detection data. The sensing units are, for example, a temperature sensing unit, an electrical conductivity sensing unit (for measuring an electrical conductivity (EC) value, which indicates the concentration of a soluble salt in a liquid), an oxidation-reduction potential sensing unit (for measuring an oxidation-reduction potential (ORP) value, which represents an oxidation-reduction capability of an aqueous solution, a dissolved oxygen sensing unit (for measuring the content of oxygen in dissolved in water), a pH value sensing unit (for measuring the acidity/alkalinity of water), and an ion-selective electrode sensing unit (an electrochemical sensor for detecting the concentration of a specified ion in a solution). The monitor 170 includes one or more sensing units, and the at least one sensing unit can be at least one selected from the above sensing units.

Further, the counterweight block 160 can be selectively in least one of the six configurations below: in the middle tube 120, outside the middle tube 120, in the lower tube 130, outside the middle tube 130, in the second connecting member 153, and outside the second connecting member 153. FIG. 2 shows an example of the counterweight block 160 configured in the second connecting member 153.

The signal transceiving module 180 generates wireless signals to be directly received by a near end or a remote end, or wireless signals directly received from a near end or a remote end. The signal transceiving module 180 can be built in with a power module, be equipped with a solar power module, or be provided with a combination of the two. The power module can be a rechargeable battery or a disposable battery. The solar power module can be configured at an upper side or around the tube of the upper tube 110. Thus, in the water quality monitoring device 100 capable of staying afloat without tiling, a part floating on the water surface can be combined with the solar power module to achieve a longer operation time.

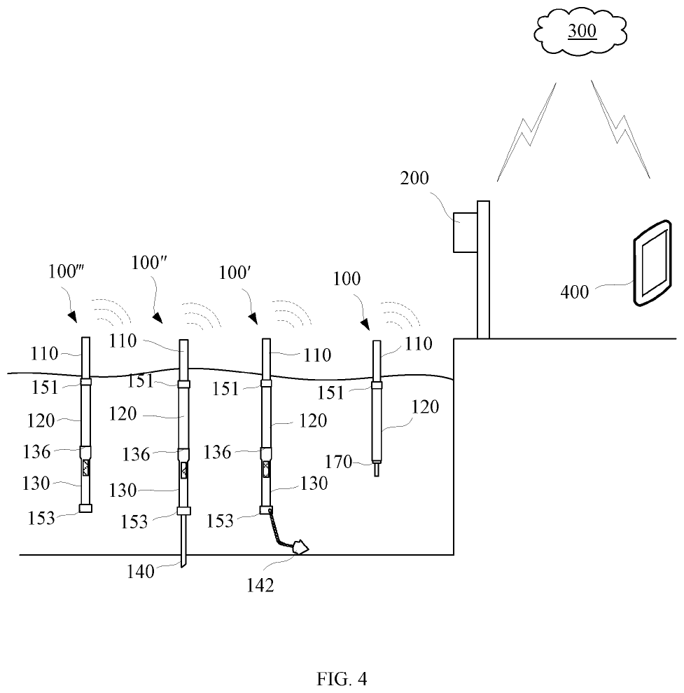

FIG. 4 shows a schematic diagram of a water quality monitoring system according to an embodiment of the present invention. In the water quality monitoring of another embodiment, for example, an anchor member 142 can be connected on the second connecting member 153 to present a water quality monitoring device 100′ as shown in FIG. 4. Using the pulling force of the anchor member 142 at the bottom of the water, the water quality monitoring device 100′ can be kept unlikely to drift to other locations. Moreover, in the water quality monitoring device of another embodiment, for example, an end tube 140 can be further connected to a lower end of the second connecting member 153. The end tube 140 has a pointed portion at a lower end thereof for easily inserting into soil at the bottom of a pond, hence securely holding a water quality monitoring device 100″ at an original location. The water quality monitoring devices 100 and 100′″ shown in FIG. 1 and FIG. 2 are also shown in FIG. 4.

Again referring to FIG. 4, the water quality monitoring devices (100, 100′, 100″ and 100′″) and a wireless router 200 can form a water quality monitoring system. The wireless router 200 is wirelessly connected to the signal transceiving modules 180 of the water quality monitoring devices (100, 100′, 100″ and 100′″), so as to obtain water quality data detected by the monitor 170.

The connection between the wireless router 200 and the signal transceiving modules 180 can be established by Wi-Fi high fidelity (Hi-Fi) transmission channels, RF transmission channels or other communication means. The wireless router 200 can be further connected to the remote server 300 by a fixed network or other connection means, and the remote server 300 then transmits monitoring data to a terminal device 400 (e.g., a communication device such as a smartphone, a tablet computer or a computer), enabling aquaculture industrialists to conveniently obtain water quality monitoring data and to take reactive measures in time in response to water quality condition alerts to reduce losses.

Further, the signal transceiving modules 180 of the water quality monitoring devices (100, 100′, 100″ and 100′″) can also directly be signal transceiving modules compliant to mobile communication technology standards, e.g., fourth generation or fifth generation of mobile communication technology standards, or even later communication technology standards. Thus, the water quality monitoring devices can be directly connected to the remote server 300, and the configuration of the wireless router 200 can be omitted.

In conclusion, the water quality monitoring device and the system thereof disclosed in the embodiments of the present invention are capable of enabling aquaculture industrialists to more conveniently and more timely monitor water quality, and to take corresponding reactive measures before or while water quality conditions deteriorate, thus reducing losses and achieving more effective protection.

The present disclosure is illustrated by various aspects and embodiments. However, persons skilled in the art understand that the various aspects and embodiments are illustrative rather than restrictive of the scope of the present disclosure. After perusing this specification, persons skilled in the art may come up with other aspects and embodiments without departing from the scope of the present disclosure. All equivalent variations and replacements of the aspects and the embodiments must fall within the scope of the present disclosure. Therefore, the scope of the protection of rights of the present disclosure shall be defined by the appended claims.

What is claimed is:

1. A water quality monitoring device, comprising:
an upper tube, having an opening at a lower end thereof;
a middle tube, having an opening at each of upper and lower ends thereof;
a first connecting member, having an upper end thereof connected to the opening at the lower end of the upper tube, and a lower end thereof connected to the opening at the upper end of the middle tube;
a monitor, having a signal transmission wire sequentially passing an interior of the middle tube and the first connecting member, the monitor being in either one of two configurations of: configured at a lower end at the interior of the middle tube and protruding out of the opening at the lower end of the middle tube, or configured at an exterior of the middle tube and connected to one end of the signal transmission wire, the end of the signal transmission wire being fixed at the lower end of the interior of the middle tube; and
a signal transceiving module, installed at an interior of the upper tube, coupled to the signal transmission wire.

2. The water quality monitoring device according to claim 1, further comprising a sealing ring between the first connecting member and the middle tube, wherein an accommodating space is defined at the interior of the middle tube by the first connecting member and the monitor configured at the lower end of the middle tube so as to preserve gas in the accommodating space therein.

3. The water quality monitoring device according to claim 2, further comprising:
a lower tube, having an opening at an upper end thereof, at least one window connecting to an interior thereof on a tube wall thereof, and a dilated opening matching an outer diameter of the middle tube at the upper end thereof; and
a second connecting member, having an upper end thereof connected to a lower end of the lower tube.

4. The water quality monitoring device according to claim 3, further comprising:
a counterweight block, being in at least one of six configurations of: in the middle tube, outside the middle tube, in the lower tube, outside the lower tube, in the second connecting member and outside the second connecting member.

5. The water quality monitoring device according to claim 4, wherein a position at which the counterweight block is configured causes a center of gravity of the water quality monitoring system to locate below a middle of the water quality monitoring device, and simultaneously causes an antenna portion of the signal transceiving module to be revealed above a water surface when the water quality monitoring device is deployed in water.

6. The water quality monitoring device according to claim 3, wherein the lower tube comprises a corresponding net mask covering the at least one window.

7. The water quality monitoring device according to claim 1, wherein the monitor comprises at least one sensing unit, and the at least one sensing unit is at least one selected from a temperature sensing unit, an electrical conductivity sensing unit, an oxidation-reduction potential sensing unit, a dissolved oxygen sensing unit, a pH value sensing unit and an ion-selective electrode sensing unit.

8. The water quality monitoring device according to claim 1, further comprising a solar power module provided at an upper side or around a tube of the upper tube, and the solar power module is coupled to the signal transmission wire.

9. The water quality monitoring device according to claim 3, further comprising an end tube having a pointed portion at a lower end thereof, an upper end of the end tube connected to a lower end of the second connecting member.

10. The water quality monitoring device according to claim 3, wherein the second connecting member is connected with an anchor member.

11. A water quality monitoring system, connected to a remote server to provide water quality data to a terminal device, the water quality monitoring system comprising:
a plurality of water quality monitoring devices of claim 1; and
a wireless router, wirelessly connected to the water quality monitoring devices so as to obtain water quality data detected by the monitors of the water quality monitoring devices.

12. The water quality monitoring system according to claim 11, wherein the signal transceiving modules of the water quality monitoring devices are radio-frequency (RF) transceiving modules to thereby establish RF communication with the wireless router.

13. A water quality monitoring system, connected to a remote server to provide water quality data to a terminal device, the water quality monitoring system comprising:
a plurality of water quality monitoring devices of claim 1, wherein the signal transceiving modules of the water quality monitoring devices are signal transceiving modules compliant to mobile communication technology standards.
